(19) United States
(12) Patent Application Publication
(10) Pub. No.: US 2003/0135059 A1
(43) Pub. Date: Jul. 17, 2003

(54) Liquid epoxy compound and process for preparing the same

(76) Inventors: Yasufumi Matsumura, Chiba (JP); Hiromi Komiyama, Chiba (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 10/322,637

(22) Filed: Dec. 19, 2002

(30) Foreign Application Priority Data
Dec. 20, 2001 (JP) 2001-387408

(51) Int. Cl. C07D 303/00

(52) U.S. Cl. 549/512

(57) ABSTRACT

A liquid trifunctional epoxy compound with a total chlorine content of 100 ppm or less represented by general formula (1) is obtained by epoxidizing a triallyl ether represented by general formula (2) with a peroxide and the liquid epoxy compound is useful as a diluent in a variety of applications, particularly useful as a liquid encapsulating material in electronic applications:

R[—(OCH₂CH₂)ₙ—OG]₃ (1)

R[—(OCH₂CH₂)ₙ—O—CH₂—CH═CH₂]₃ (2)

(wherein R is a trivalent hydrocarbon group in which 3 different carbon atoms participate in bond formation or, more particularly, a straight-chain or branched hydrocarbon group containing 3-10 carbon atoms, a cycloalkane group containing 6 carbon atoms or a hydrocarbon group containing 7-20 carbon atoms and consisting of hydrocarbon chains and cycloalkane rings, n is 0, 1, or 2 and G is glycidyl group).

### FIELD OF THE INVENTION

This invention relates to a trifunctional liquid epoxy compound which is low in viscosity and easy to work with and cures readily by heating with excellent mechanical and electrical properties. The liquid epoxy compound is useful as a diluent in many applications, particularly, as liquid materials for electronic materials.

### BACKGROUND OF THE INVENTION

A liquid epoxy compound (also referred to as epoxy resin) is used as a binder in a variety of applications on account of its solubility characteristics and good mechanical properties. However, in the cases where the mechanical properties need to be improved, a generally adopted technique of increasing the amount of a filler and the like in the formulation would increase the viscosity and deteriorate the workability. To prevent this, the viscosity of the ingredients of a binder needs to be reduced. As for an epoxy compound that constitutes the main ingredient of a binder, maintaining or improving the mechanical properties of the cured product is generally mutually exclusive to reducing the viscosity and, to solve this problem, a liquid epoxy resin has been used as a reactive diluent. Examples of such liquid epoxy resins are ethylene glycol glycidyl ether, propylene glycol glycidyl ether, glycerin glycidyl ether, trimethylolpropane glycidyl ether, cyclohexanedimethanol glycidyl ether, phenyl glycidyl ether, resorcin glycidyl ether and xylylene glycol glycidyl ether (JP4-53821 A).

Aliphatic epoxy resins selected from alicyclic and aliphatic chain epoxy resins are in the category exhibiting the lowest viscosity and produce an excellent diluting effect. Of these epoxy resins, glycidyl ethers of trihydroxy compounds such as glycerin and trimethylolpropane are trifunctional leading to a tridimensional structure and they are excellent reactive diluents in respect to the mechanical properties. However, an epoxy compound obtained by the epoxidation of an aliphatic alcohol with epichlorohydrin contains the unreacted OH groups because of poor reactivity of the alcoholic hydroxyl group. In particular, the glycidyl ether of a polyhydric alcohol contains residual OH groups and the ether becomes difficult to separate and purify. Moreover, in the case of an aliphatic epoxy compound, the amount of residual chlorine reaches a high level due to a secondary reaction which occurs during the epoxidation reaction with epichlorohydrin. Application of these epoxy compounds to electronic materials adversely affects the electrical reliability. As a technique for reducing the chlorine content of aliphatic epoxy compounds, the use of an alkali metal hydroxide in a large excess is described in JP59-206429 A, but the yield is low and the total chlorine content is as high as 3,000 ppm or more. The use of solid alkali metal hydroxide is described in JP 1-151567 A, but the procedure to conduct the reaction is complicated and the total chlorine content cannot be reduced sufficiently.

Epoxy compounds based on phenols such as phenol novolaks are relatively low in chlorine content, but the viscosity increases markedly as the number of functional groups increases and those epoxy compounds which contain 3 functional groups become highly viscous and cannot be expected to be effective as a diluent.

Keeping pace with improved performance of digital equipment such as larger capacity for data storage and higher speed in data processing in recent years, achievement of a greater density for wiring boards and packaging of components and innovation in the packaging technology of semiconductor devices have widened an area for application of liquid encapsulating materials and there has arisen a strong demand for further improvement of reactive diluents to be used for liquid encapsulating materials.

### DISCLOSURE OF THE INVENTION

An object of this invention is to provide a high-purity liquid epoxy compound which maintains a low viscosity suitable for use as a diluent for a variety of applications, particularly as a liquid encapsulating material for electronic materials and can be improved in reliability and to provide a process for preparing said liquid epoxy compound.

This invention relates to a trifunctional epoxy compound which is represented by general formula (1)

R[—(OCH₂CH₂)ₙ—OG]₃ (1)

(wherein R is a trivalent hydrocarbon group in which 3 different carbon atoms participate in bond formation or, more particularly, a straight-chain or branched hydrocarbon group containing 3-10 carbon atoms, a cycloalkane group containing 5-6 carbon atoms or a hydrocarbon group containing 7-20 carbon atoms and consisting of hydrocarbon chains and cycloalkane rings, n is 0, 1 or 2 and G is glycidyl group) and has a total chlorine content of 100 ppm or less.

This invention further relates to the aforementioned liquid epoxy compound which is obtained by epoxidizing a triallyl ether represented by general formula (2) with a peroxide.

R[—(OCH₂CH₂)ₙ—O—CH₂CH═CH₂]₃ (2)

Wherein R and n are as defined in general formula (1).

Still more, this invention relates to a process for preparing the aforementioned liquid epoxy compound which comprises epoxidizing the aforementioned triallyl ether represented by the aforementioned general formula (2) with a peroxide.

The group R in general formulas (1) and (2) is preferably a trivalent hydrocarbon group represented by the following formulas (a), (b), (c) or (d)

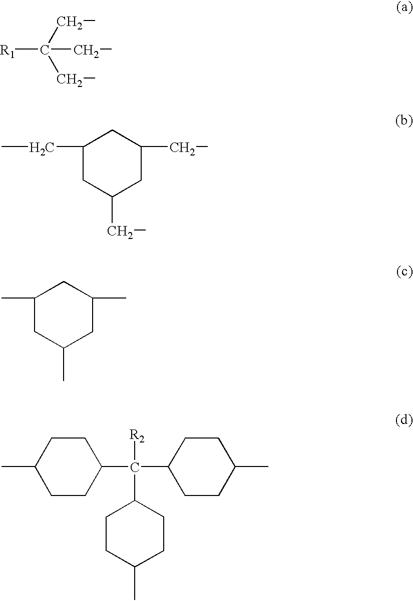

Wherein R₁ and R₂ is a hydrogen atom or an alkyl group containing 1-4 carbon atoms.

### DESCRIPTION OF THE PREFFERED EMBOBODIMENTS

The liquid epoxy compound of this invention is represented by the aforementioned general formula (1). The liquid epoxy compound of this invention may also be called a liquid epoxy resin. The epoxy compounds represented by general formula (1) can be synthesized, for example, by allylating a trihydroxy compound represented by general formula (3) to a triallyl ether represented by general formula (2) and epoxidizing the triallyl ether.

R[—(OCH₂CH₂)ₙ—OH]₂ (3)

R[—(OCH₂CH₂)ₙ—O—CH₂CH═CH₂]₃ (2)

R[—(OCH₂CH₂)ₙ—OG]₃ (1)

Wherein R, n and G in general formulas (2) and (3) are the same as in general formula (1).

The steps for the synthesis of a liquid epoxy compound represented by general formula (1) from a trihydroxy compound are shown below for the case where n is 0 and R is represented by formula (a) in general formulas (1), (2) and (3).

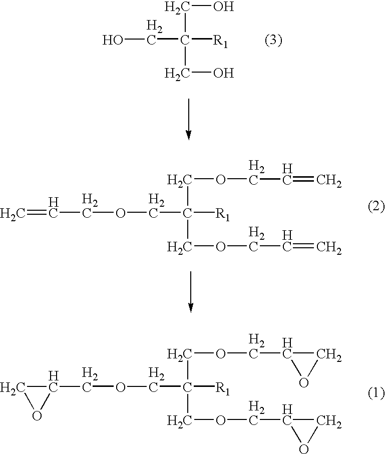

Examples of a trihydroxy compound represented by the aforementioned general formula (3) include the following: glycerol or a compound represented by general formula (3) wherein R is —CH₂—C*H—CH₂— and n is 0; trimethylolpropane or a compound represented by general formula (3) wherein R is represented by formula (a), n is 0 and R₁ is ethyl; 1,3,5-trimethylolcyclohexane or a compound represented by general formula (3) wherein R is represented by formula (b) and n is 0; 1,3,5-trihydroxycyclohexane or a compound represented by general formula (3) wherein R is represented by formula (c) and n is 0; tris(4-hydroxycyclohexyl)methane or a compound represented by general formula (3) wherein R is represented by formula (d) and n is 0; ethylene oxide-modified trimethylolpropane or a compound represented by general formula (3) wherein R is represented by formula (a), n is 1 or 2 and R₁ is ethyl. As is apparent from the aforementioned reaction equations, R and n remain unchanged in formulas (1), (2) and (3) and the triallyl ether represented by general formula (2) obtained from the trihydroxy compound represented by general formula (3) and the epoxy compound represented by general formula (1) obtained from the triallyl ether can readily be understood from the explanation of the trihydroxy compound represented by general formula (3).

Ethylene oxide-modified trimethylolpropane which is a compound represented by general formula (3) wherein R is represented by formula (a), n is 1 or 2 and R₁ is ethyl is represented by the following chemical formula or the following general formula (4) wherein n is 1 or 2.

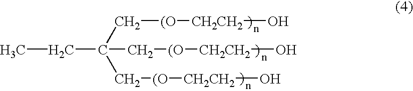

When R in the aforementioned general formulas (1) to (3) is a chain hydrocarbon group, it is preferably a saturated hydrocarbon group containing 3-10 carbon atoms. When R is a hydrocarbon group consisting of hydrocarbon chains and cycloalkane rings, it is preferably a saturated hydrocarbon group containing 7-20 carbon atoms.

The synthesis of a triallyl ether represented by general formula (2) starting from a trihydroxy compound represented by general formula (3) can be carried out as follows. The trihydroxy compound is added to a base or an aqueous solution of alkali such as sodium hydroxide, the mixture is heated at 80-90° C. for 0.2-5 hours, cooled to 40-50° C., a quaternary ammonium salt (for example, a tetraalkylammonium halide such as tetrabutylammonium bromide or a tetraarylammonum halide such as tetraphenylammonium chloride) is added as a catalyst and then allyl chloride is added in drops to the mixture at 40-50° C. Upon completion of the dropwise addition, the reaction is allowed to proceed at 40-50° C. for 2-10 hours.

A preferred base is sodium hydroxide, calcium hydroxide or potassium hydroxide. The amount of the base to be added is 5 to 25 times, preferably 6 to 20 times, the equivalent of the trihydroxy compound.

The amount of the quaternary ammonium salt to be added as a catalyst is 0.4-0.6 mole per 1 mole of the hydroxy compound. The amount of allyl chloride to be added is 4-5 moles per 1 mole of the trihydroxy compound.

Upon completion of the allylation reaction, the reaction mixture is cooled to room temperature or thereabouts, an organic solvent such as toluene is added, and the organic layer is separated, concentrated and purified by a short silica gel column to give a triallyl ether represented by the general formula (2). The purity of the triallyl ether should be made 98% or more, preferably 99% or more.

The synthesis of an epoxy compound represented by general formula (1) from the triallyl ether represented by general formula (2) prepared above can be carried out as follows. The triallyl ether is dissolved in an organic solvent such as methylene chloride, the mixture is cooled to 0° C. or thereabouts, a peracid such as m-chloroperbenzoic acid and peracetic acid or a peroxide-based oxidizing agent such as hydrogen peroxide is added and the epoxidation reaction is allowed to proceed at room temperature for 8-10 hours.

Upon completion of the epoxidation reaction, the oxidizing agent is neutralized by an aqueous alkaline solution, the reaction mixture is treated with an organic solvent such as toluene and the organic layer is concentrated to yield an epoxy compound represented by general formula (1).

The amount of the peroxide such as m-chloroperbenzoic acid to be added as an oxidizing agent is 4-6 times the equivalent of the allyl ether or 4-6 moles per 1 mole of the allyl ether. A preferred peroxide is peracid.

An organic solvent to be used in the epoxidation reaction is preferably a halogenated hydrocarbon such as methylene chloride and a hydrocarbon such as hexane and toluene and methylene chloride is particularly preferable. The triallyl ether is dissolved in the solvent to a concentration in the range of 0.2-0.6 mol/L, preferably 0.3-0.5 mol/L.

The liquid epoxy compound obtained in this manner is a product of high purity because of the presence of a small amount of unreacted substances. It is therefore possible to control the epoxy equivalent (Eq) nearly at the theoretical value. Preferably, the ratio of the epoxy equivalent (Eq) of the product liquid epoxy compound to the value obtained by dividing the molecular weight (M) of the liquid epoxy compound calculated from-its chemical formula by the number of epoxy groups or 3 (Eq′=M/3) or the ratio Eq/Eq′ is in the range of 95-100%. For example, the epoxy equivalent of tris(glycidyloxymethyl)methane is in the range of 91-93 while that of tris(glycidyloxymethyl)propane is in the range of 100-102. It is also possible to control the epoxy equivalent in the desired range by selecting or mixing the starting material trihydroxy compound or mixing the product liquid epoxy compound.

The liquid epoxy compound obtained by the process of this invention contains a small amount of unreacted substances and a small amount of residual chlorine attributable to the secondary reaction of the conventional process which uses epichlorohydrin or 100 ppm or less as the total chlorine content and it is possible to reduce the chlorine content to 50 ppm or less in case the liquid epoxy compound is intended for electronic applications. The epoxy compound of this invention contains 95% or more, preferably 98% or more, more preferably 99% or more, of the epoxy compound represented by general formula (1) and a small amount of impurities such as chlorine compounds.

The liquid epoxy compound of this invention can be cured by incorporating a known epoxy curing agent such as an acid anhydride and an imidazole derivative. It can also be incorporated to other curable compositions as a reactive diluent.

### EXAMPLES

Using a trihydroxy compound represented by general formula (3) as a starting material, a triallyl ether represented by general formula (2) was synthesized, separated and purified, the triallyl ether obtained was used to synthesize an epoxy compound represented by general formula (1) and the epoxy compound was separated and purified. In the following examples, a description is given of the synthesis of a triallyl ether (allylation), the purification of the triallyl ether, the synthesis of an epoxy compound (epoxidation) and the purification of the epoxy compound in this order.

### Example 1

In a 1-L four-necked glass flask equipped with a stirrer, a thermometer, an inlet for nitrogen gas, a dropping device and a condenser was placed 53.67 g of trimethylolpropane, the air in the flask was replaced with nitrogen, 352 ml (25 mol/L) of an aqueous solution of sodium hydroxide was added and the mixture was heated to 80° C. and stirred at this temperature for 1 hour. Thereafter, the mixture was cooled to 40° C., 51.58 g of tetrabutylammonium bromide was added and then 122.2 g of allyl chloride was added in drops to the mixture over a period of 1 hour while keeping the mixture at approximately 40° C. on a water bath. The allylation reaction was continued for another 5 hours to completion.

Upon completion of the reaction, 500 ml of toluene was added to the reaction mixture, the organic layer was separated washed by an aqueous solution of sodium chloride until the washing became neutral. Thereafter, the washed organic layer was dried over magnesium sulfate and concentrated. The concentrate was purified by 500 g of silica gel and toluene to give 76.31 g of 1,1,1-tri(allyloxymethyl)propane. The yield was 100% and the purity (by gas chromatography) was 99%.

In a 3-L four-necked glass flask equipped with a stirrer, a thermometer, an inlet for nitrogen gas and a condenser was placed 1,200 ml of methylene chloride, the air inside the flask was replaced with nitrogen and 76.31 g of the 1,1,1-tri(allyloxymethyl)propane obtained above was added. Thereafter, the reaction system was cooled to 0° C. in an ice bath, 201.9 g of m-chloroperbenzoic acid was added in 4 portions, and the mixture was stirred at room temperature for 8 hours until the epoxidation reaction was completed.

Upon completion of the reaction, the reaction mixture was cooled to 10° C. in an ice bath, 1 N aqueous sodium thiosulfate solution was added, the mixture was allowed to return to room temperature and stirred for 1 hour. Following this, 1,200 ml of methylene chloride was added to the mixture, the organic layer was separated, washed with an aqueous solution of sodium hydroxide (0.5N) and then washed with an aqueous solution of sodium chloride until the washing became neutral. Thereafter, the washed organic layer was dried over magnesium sulfate and stripped of the methylene chloride to give 93.31 g of 1,1,1-tri(glydicyloxymethyl)propane. The yield was 80% and the purity was 99%.

The epoxy compound obtained was a colorless liquid with a viscosity of 57 mPa·sec (23° C.), an epoxy equivalent of 101 and a total chlorine content of 32 ppm.

### Example 2

The allylation reaction was carried out as in Example 1 except using 42.07 g of 1,1,1-tri(methylol)methane, 40.43 g of tetrabutylammonium bromide and 95.79 g of allyl chloride. After the allylation reaction, the product was separated and purified as in Example 1.

As a result, 59.80 g of 1,1,1-tri(allyloxymethyl)methane was obtained. The yield was 100% and the purity was 99%.

The epoxidation reaction was then carried out as in Example 1 by the use of 59.80 g of the 1,1,1-tri(allyloxymethyl)methane obtained above and 158.3 g of m-chloroperbenzoic acid. After the epoxidation reaction, the product was separated and purified as in Example 1.

As a result, 73.14 g of 1,1,1-tri(glycidyloxymethyl)methane was obtained. The yield was 85% and the purity was 99%. The product was a colorless liquid with a viscosity of 55 mPa·sec (23° C.), an epoxy equivalent of 92 and a total chlorine content of 26 ppm.

### Example 3

The allylation reaction was carried out as in Example 1 by the use of 189.5 g of diethylene oxide-modified trimethylolpropane which corresponds to a compound represented by general formula (4) wherein n is 2, 400 ml of an aqueous solution of sodium hydroxide, 51.58 g of tetrabutylammonium bromide and 122.2 g of allyl chloride.

Upon completion of the reaction, the product was purified as in Example 1 to give 237.0 g of triallyloxydiethyleneoxidemethylenepropane. The yield was 100% and the purity was 99%.

Thereafter, 271.5 g of the triallyloxydiethyleneoxidemethylenepropane was epoxidized with 201.9 g of m-chloroperbenzoic acid as in Example 1.

Upon completion of the reaction, the product was purified as in Example 1 to give 192.1 g of triglycidyloxydiethyleneoxidemethylenepropane. The yield was 75% and the purity was 99%. The epoxy compound thus obtained was a colorless liquid with an epoxy equivalent of 242 and a total chlorine content of 30 ppm.

### Example 4

The allylation reaction was carried out as in Example 1 by the use of 124.6 g of the tris(hydroxy-diethyleneoxide-methyl)propane which corresponds to a compound represented by general formula (4) wherein n is 1, 370 ml (25 mol/L) of an aqueous solution of sodium hydroxide, 51.58 g of tetrabutylammonium bromide and 122.2 g of allyl chloride.

Upon completion of the reaction, the product was purified as in Example 1 to give 172.5 g of triallyloxyethyleneoxidemethylenepropane. The yield was 100% and the purity was 99%.

The epoxidation reaction of 172.0 g of the triallyloxyethyleneoxidemethylenepropane obtained above with 201.9 g of m-chloroperbenzoic acid was carried out as in Example 1.

Upon completion of the reaction, the product was purified as in Example 1 to give 153.0 g of tris(glycidyloxyethyleneoxidemethyl)propane. The yield was 80% and the purity was 99%. The product thus obtained was a colorless liquid with an epoxy equivalent of 160 and a total chlorine content of 25 ppm.

### Example 5

The allylation reaction was carried out as in Example 1 by the use of 52.8 g of 1,3,5-trihydroxycyclohexane, 350 ml (25 mol/L) of an aqueous solution of sodium hydroxide, 51.58 g of tetrabutylammonium bromide and 122.2 g of allyl chloride.

Upon completion of the reaction, the product was purified as in Example 1 to give 100.1 g of 1,3,5-triallyloxycyclohexane. The yield was 100% and the purity was 99%.

The epoxidation reaction was then carried out as in Example 1 by the use of 100.0 g of the 1,3,5-triallyloxycyclohexane obtained above and 201.9 g of m-chloroperbenzoic acid.

Upon completion of the reaction, the product was purified as in Example 1 to give 107.5 g of 1,3,5-triglycidyloxycyclohexane. The yield was 90% and the purity was 99%. The product obtained was a colorless liquid with an epoxy equivalent of 101 and a total chlorine content of 26 ppm.

### Example 6

The allylation reaction was carried out as in Example 1 by the use of 69.7 g of 1,3,5-trimethylolcyclohexane, 350 ml (25 mol/L) of an aqueous solution of sodium hydroxide, 51.58 g of tetrabutylammonium bromide and 122.2 g of allyl chloride.

Upon completion of the reaction, the product was purified as in Example 1 to give 117.2 g of 1,3,5-triallyloxylmethyl-cyclohexane. The yield was 100% and the purity was 99%.

The epoxidation reaction was then carried out as in Example 1 by the use of 117.2 g of the 1,3,5-triallyloxylmethyl-cyclohexane obtained above and 201.9 g of m-chloroperbenzoic acid.

Upon completion of the reaction, the product was purified as in Example 1 to give 123.2 g of 1,3,5-triglycidyloxymethyl-cyclohexane. The yield was 90% and the purity was 99%. The product obtained was a colorless liquid with an epoxy equivalent of 114 and a total chlorine content of 28 ppm.

### Example 7

The allylation reaction was carried out as in Example 1 by the use of 129.0 g of tris(4-hydroxycyclohexyl)methane, 370 ml (25 mol/L) of an aqueous solution of sodium hydroxide, 51.58 g of tetrabutylammonium bromide and 122.2 g of allyl chloride.

Upon completion of the reaction, the product was purified as in Example 1 to give 177.0 g of tris(allyloxycyclohexyl)methane. The yield was 100% and the purity was 99%.

The epoxidation reaction was then carried out as in Example 1 by the use of 1,200 ml of methylene chloride, 117.0 g of the tris(allyloxycyclohexyl)methane obtained above and 201.9 g of m-chloroperbenzoic acid.

Upon completion of the reaction, the product was purified as in Example 1 to give 157.6 g of tris(4-glycidyloxycyclohexyl)methane. The yield was 80% and the purity was 99%. The epoxy compound obtained was a colorless liquid with an epoxy equivalent of 164 and a total chlorine content of 32 ppm.

We claim:

1. A trifunctional epoxy compound which is represented by general formula (1)
R[—(OCH₂CH₂)ₙ—OG]₃ (1)
(R is a trivalent hydrocarbon group in which 3 different carbon atoms participate in bond formation, wherein the trivalent hydrocarbon group is a straight-chain or branched chain hydrocarbon group containing 3-10 carbon atoms, a cycloalkane group containing 5-6 carbon atoms, or a hydrocarbon group containing 7-20 carbon atoms and consisting of hydrocarbon chains and cycloalkane rings, n is 0, 1 or 2 and G is glycidyl group) and has a total chlorine content of 100 ppm or less.

2. A liquid epoxy compound as described in claim 1 wherein the total chlorine content is 50 ppm or less.

3. A liquid epoxy compound as described in claim 1, said liquid epoxy compound being obtained by epoxidizing a triallyl ether represented by general formula (2)
R[—(OCH₂CH₂)ₙ—O—CH₂CH═CH₂]₃ (2)
(R is a trivalent hydrocarbon group in which 3 different carbon atoms participate in bond formation, wherein the trivalent hydrocarbon group is a straight-chain or branched chain hydrocarbon group containing 3-10 carbon atoms, a cycloalkane group containing 5-6 carbon atoms or a hydrocarbon group containing 7-20 carbon atoms and consisting of hydrocarbon chains and cycloalkane rings, n is 0, 1 or 2) with a peroxide.

4. A process for preparing a liquid epoxy compound described in claim 1 which comprises epoxidizng a triallyl ether represented by general formula (2)
R[—(OCH₂CH₂)ₙ—O—CH₂CH═CH₂]₃ (2)
(R is a trivalent hydrocarbon group in which 3 different carbon atoms participate in bond formation, wherein the trivalent hydrocarbon group is a straight-chain or branched chain hydrocarbon group containing 3-10 carbon atoms, a cycloalkane group containing 5-6 carbon atoms or a hydrocarbon group containing 7-20 carbon atoms and consisting of hydrocarbon chains and cycloalkane rings, n is 0, 1 or 2) with a peroxide.

5. A process for preparing a liquid epoxy compound as described in claim 4 wherein the triallyl ether represented by general formula (2) is obtained by the reaction of a trihydroxy compound represented by general formula (3)
R[—(OCH₂CH₂)ₙ—OH] (3)
(R is a trivalent hydrocarbon group in which 3 different carbon atoms participate in bond formation, wherein the trivalent hydrocarbon group is a straight-chain or branched chain hydrocarbon group containing 3-10 carbon atoms, a cycloalkane group containing 5-6 carbon atoms or a hydrocarbon group containing 7-20 carbon atoms and consisting of hydrocarbon chains and cycloalkane rings, n is 0, 1 or 2) with an allyl halide.

6. A process for preparing a liquid epoxy compound as described in claim 4 wherein R in general formulas (1) and (2) is a trivalent hydrocarbon group represented by the following formulas (a), (b), (c) or (d) 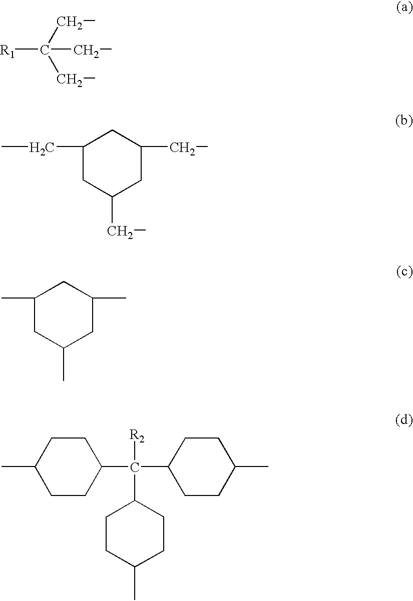
(R₁ and R₂ is a hydrogen atom or an alkyl group containing 1-4 carbon atoms).
